(19) United States
(12) Patent Application Publication
(10) Pub. No.: US 2003/0232337 A1
(43) Pub. Date: Dec. 18, 2003

(54) Detection of Staphylococcus spp.

(76) Inventors: Lu-Yieng Liu, Hsinchu (TW); Kan-Hung Lee, Hsinchu (TW); Harn-Jing Terng, Hsinchu (TW)

Correspondence Address: FISH & RICHARDSON PC, 225 FRANKLIN ST, BOSTON, MA, 02110, US

(21) Appl. No.: 10/164,758

(22) Filed: Jun. 7, 2002

(51) Int. Cl. C12Q 1/68; C07H 21/04

(52) U.S. Cl. 435/6; 536/23.7; 536/24.3

(57) ABSTRACT

A novel nucleic acid containing an oligo-nucleotide selected from a member of the group consisting of SEQ ID NOs: 1-25 and sequences complementary to SEQ ID NOs: 1-25. The nucleic acid is 10-1000 nucleotides in length. Also disclosed is a method of detecting Staphylococcus spp.

### BACKGROUND

Traditionally, detection of a microorganism requires time-consuming growth of the microorganism in a culture medium, followed by its isolation and identification. The entire process usually takes 24-48 hours. Many methods for rapid detection of microorganisms have recently been developed, including miniaturized biochemical analyses, antibody- and DNA-based tests, and modified conventional assays.

Staphylococci bacteria are the causative agents of many opportunistic human and animal infections. Accurate and rapid identification of Staphylococcus spp. is conducive to diagnosing and treating such infections.

### SUMMARY

The present invention relates to specific nucleic acid sequences selected from the Staphylococcus spp. gap gene region for detecting Staphylococcus spp.

In one aspect, this invention features a novel nucleic acid containing an oligo-nucleotide selected from a member of the group consisting of SEQ ID NOs: 1-25 and sequences complementary to SEQ ID NOs:1-25, wherein the nucleic acid is 10-1000 (e.g., 10-500, 10-200, 10-50, and 10-20) nucleotides in length. The nucleic acid can simply be a member of the group consisting of SEQ ID NOs: 1-25 and sequences complementary to SEQ ID NOs: 1-25. These nucleic acids can be used as sequencing primers, PCR primers and hybridization probes.

In another aspect, this invention features a pair of amplification primers: one primer contains an oligo-nucleotide selected from a member of the group consisting of SEQ ID NOs:28-52, and the other primer contains an oligo-nucleotide selected from a sequence complementary to the member. Each primer is 14-40 (e.g., 14-30 and 14-20) nucleotides in length. These primers can be used to amplify a DNA template prepared from Staphylococcus spp.

Also within the scope of this invention is a method of detecting a target Staphylococcus species. The method involves (1) providing a sample having a nucleic acid from an unknown microorganism; (2) amplifying the nucleic acid with a pair of primers: one primer contains an oligo-nucleotide selected from a member of the group consisting of SEQ ID NOs:28-52, and the other primer contains an oligo-nucleotide selected from a sequence complementary to the member. Each primer is 14-40 nucleotides in length; and (3) detecting an amplification product. Detection of the amplification product indicates the presence of the target Staphylococcus species. In one example, the detecting step includes hybridizing the amplification product to a nucleic acid probe that is 10-1000 nucleotides in length and contains a sequence selected from a member of the group consisting of SEQ ID NOs: 1-27 and sequences complementary to SEQ ID NOs: 1-27. The nucleic acid probe can simply be a member of the group consisting of SEQ ID NOs: 1-27 and sequences complementary to SEQ ID NOs: 1-27.

Further within the scope of this invention is a kit for detecting Staphylococcus spp. The kit contains one or more of the nucleic acids described above. It can also include other components such as a DNA polymerase, a PCR buffer, or a solid support on which one or more of the above-described probes are immobilized.

The present invention provides a fast, accurate, and sensitive method for Staphylococcus spp. detection and typing. The details of one or more embodiments of the invention are set forth in the accompanying description below. Other advantages, features, and objects of the invention will be apparent from the detailed description, and from the claims.

### DETAILED DESCRIPTION

The present invention is based on the discovery that the Staphylococcus spp. gap gene region can be used for identification of specific Staphylococcus species. The gap gene regions of 27 Staphylococcus spp. are amplified using a primer pair (GF-1: 5′-atggttttggtagaattggtcgttta-3′ and GR-2: 5′-gacatttcgttatcataccaagctg-3′; Yugueros et al., 2000, J. Clinical Microbiology 38: 4351-4355) located in the conserved region of the gap gene. Sequences of the amplified DNA fragments are shown below:

(1) Staphylococcus arlettae

| | | | |
|---|---|---|---|
| 1 | atggttttggtagaattggtcgtttagcatttagaagaattcaaaacgttgatggaatcg | 60 | (SEQ ID NO:1) |
| 61 | acgttgtagcagtaaacgacttaacagatgatgaaatgttagcacacttattaaaatatg | 120 | |
| 121 | acactatgcaaggacgtttcacaggagaagttgaagtagaaaatgacggtttccgtgtaa | 180 | |
| 181 | atggtcaagaagttaaatcattctcagaaccagatccaaataaattaccatggggcgact | 240 | |
| 241 | tagatatcgatgttgttttagaacgtactggtttattcgcagacaaagataaagcatcag | 300 | |
| 301 | ctcatatcgacgcaggtgcgaaaaaagtattaatctctgctccagcatcaggtgatttaa | 360 | |
| 361 | aaacaatcgtattcaacactaaccacaatgaattagatggttctgaaacagttgtatcag | 420 | |
| 421 | gtgcttcatgtactactaactcattagctccagttgctaaagtattaaacgatgacttcg | 480 | |
| 481 | gtttagtagaaggtttaatgactactatccatgcttacactggtgaccaaagcacacaag | 540 | |
| 541 | acgctcctcacagaaaaggtgacaaacgtcgtgcgcgtgcagctgctgaaaacattattc | 600 | |
| 601 | ctaactcaacaggtgctgctaaagcaatcggtttagttattcctgaaatcgatggtaaat | 660 | |
| 661 | tagatggtggcgctcaacgtgtaccagtagctactggttcattaactgaattaacagttg | 720 | |
| 721 | tattagacaaagaagtaactgttgaagacgttaatagtgcaatgaaaaatgcttcaaacg | 780 | |
| 781 | aatcatttggttacactgaagacgaaatcgtttcttctgacgtaatcggtatgacttacg | 840 | |
| 841 | gttcattattcgatgctacacaaactcgtgtaatgactgttggtaaccaacaaatggtta | 900 | |
| 901 | aagtagcagcttggtatgataacgaaatgtc | | |

(2) Staphylococcus auricularia

| | | | |
|---|---|---|---|
| 1 | atggttttggtagaattggtcgtttagcattcagaagaattcaaaatgttgaaggcattg | 60 | (SEQ ID NO:2) |
| 61 | acgtagtagcagttaatgacttaacagatgacgatatgttagcacacttattaaaatacg | 120 | |
| 121 | acacaatgcaaggtcgtttcacaagtgaagttgaaatcgtagaaggcggtttccgcgtta | 180 | |
| 181 | acggtcaagaagtgaaatcattcgacgaaccagatccaagcaaattaccttggaaagact | 240 | |
| 241 | tagacatcgacgttgtacttgaatgtactggtttattcacagacaaagaaaaagcagaag | 300 | |
| 301 | cacacatcgacgcaggtgctaaaaaagtattaatctctgcaccagctaaaggtgacgtta | 360 | |
| 361 | aaacagttgtttataacactaaccacgatacacttgatggtacagaaacagttgtttcag | 420 | |
| 421 | gtgcttcatgtacaacaaactcattagcaccagttgctaaagtacttaacgatgaattcg | 480 | |
| 481 | gtttagtagaaggattcatgactacaatccacgcatacactggtgaccaaaatacacaag | 540 | |
| 541 | atgcaccacacaaaaaaggtgacaaacgccgtgcacgtgcagctgctcaaaacatcatcc | 600 | |
| 601 | ctaactctacaggtgctgcaaaagcaatcggtaaagttatcccagaaatcgaaggtaaat | 660 | |
| 661 | tagacggcggtgcacaacgtgtccctgttgctactggttcattaacagaattaacagtag | 720 | |
| 721 | tattagaaaaagatgtttcaatcgaagacgtaaacaacgcaatgaaaaatgcatcaactg | 780 | |
| 781 | aatcattcggttacacagatgacgaaatcgtttcttctgacgtcatcggtatgacttatg | 840 | |
| 841 | gttcattattcgacgcaactcaaactcgtgtcatgactgttggcgatcaccaattaatca | 900 | |
| 901 | aagttgcagcttggtatgataacgaaatgtc | | |

(3) Staphylococcus caprae

| | | | |
|---|---|---|---|
| 1 | atggttttggtagaattggtcgtttagcattcagaagaatccaagatgtagaaggtctta | 60 | (SEQ ID NO:3) |
| 61 | aagtagtagcagttaacgacttaacagacgatgaaatgttagctcatttattaaaatatg | 120 | |
| 121 | acactatgcaaggacgcttcactggagaagttgaagttatcgaaggcggattccgcgtaa | 180 | |
| 181 | acggtaaagaaattaaatcattcgacgaaccagatgcaaacaaattaccttggggtgacc | 240 | |
| 241 | ttgacatcgatgtagtattagaatgtactggtttctacactgataaagataaagcacaag | 300 | |
| 301 | cacatatcgatgcaggtgctaaaaaagtattaatctcagctccagctactggtgatttaa | 360 | |
| 361 | aaacaatcgtattcaatactaaccatgacgaattagatggttcagaaacagttgtatcag | 420 | |
| 421 | gtgcttcttgtacaactaactcattagctcctgttgctaaagtattaaatgatgaattcg | 480 | |
| 481 | gtttagttgaaggtttaatgacaactatccacgcttacacaggtgaccaaaatactcaag | 540 | |
| 541 | acgcacctcacagaaaaggtgacaaacgtcgtgctcgtgcagcagcagaaaacattatcc | 600 | |
| 601 | ctaactcaacaggtgctgctaaagcaatcggtaaagtaattcctgaaatcgacggtaaat | 660 | |
| 661 | tagacggtggtgcacaacgtgttccagttgctacaggttcattaacagaattaactgtag | 720 | |
| 721 | tattagaaaaacaagatgtaactgctgatcaagttaacgaagcaatgaaacaagcttctg | 780 | |
| 781 | acgaatcattcggttacactgaagatgaaatcgtatcttctgacgttgtaggtatcactt | 840 | |
| 841 | atggttcattattcgatgcaactcaaactcgtgtaatgactgttggtgaccgtcaattag | 900 | |
| 901 | ttaaagtagcagcttggtatgataacgaaatgtc | | |

(4) Staphylococcus capitis

| | | | |
|---|---|---|---|
| 1 | atggttttggtagaattggtcgtttagcattcagaagaatccaagatgtagaaggtctag | 60 | (SEQ ID NO:4) |
| 61 | aagtagttgcagttaacgacttaacagatgatgaaatgttagctcacttattaaaatatg | 120 | |
| 121 | acactatgcaaggtcgcttcactggagaagttgaagtcatcgatggtggattccgtgtta | 180 | |
| 181 | acggtaaagaaattaaatcattcgatgaaccagatgcaagcaaattaccttggggagacc | 240 | |
| 241 | tagacatcgacgtagtattagaatgtactggtttctacactgacaaagataaagcacaag | 300 | |
| 301 | ctcacatcgatgcaggtgctaaaaaagtattaatctctgctccagctacaggtgatttaa | 360 | |
| 361 | aaacaatcgtattcaacactaaccatgatgaattagatggttctgaaacagttgtatcag | 420 | |
| 421 | gtgcttcttgtacaactaactcattagcaccagttgctaaagttttaaatgatgagtttg | 480 | |
| 481 | gtttagttgaaggtttaatgactactatccacgcttatactggtgaccaaaatactcaag | 540 | |
| 541 | atgctcctcacagaaaaggcgacaaacgtcgtgctcgtgcagcagcagaaaacattatcc | 600 | |
| 601 | ctaactctacaggtgctgctaaagcaattggtaaagttattccagaaatcgatggtaaat | 660 | |
| 661 | tagacggtggagctcaacgtgttccagttgcaacaggttcattaactgaattaacagtag | 720 | |
| 721 | tattagataaacaagatgtaacagctgatcaagtaaatgaagcaatgaaacaagcttctg | 780 | |
| 781 | acgaatcattcggttacactgaagatgaaatcgtaccttctgacgttgtaggtatgactt | 840 | |
| 841 | atggttcattattcgacgctactcaaactcgtgtaatgactgttggtgaccgtcaattag | 900 | |
| 901 | ttaaagttgcagcttggtatgataacgaaatgtc | | |

(5) Staphylococcus carnosus

| | | | |
|---|---|---|---|
| 1 | atggttttggtagaattggtcgtttagcatttagaagaattcaagatgttgaaggtatcg | 60 | (SEQ ID NO:5) |
| 61 | atgtagtagcagtaaacgacttaacagatgatgaaatgttagcacacttattaaaatacg | 120 | |
| 121 | atacaatgcaaggacgtttcactgaagaagttgaagttgtagatggcggattccgcgtga | 180 | |
| 181 | atggtaaagaagttaaatcattcgaagaaccagatgcaagcaaattaccttggaaagatt | 240 | |
| 241 | tagacattgatgttgtattagaatgtacaggtttctatacaagtgaagaaaaagcaaaag | 300 | |
| 301 | ctcatatcgatgcaggcgctaaaaaagtattaatttcagcaccagctactggtgatgtta | 360 | |
| 361 | aaacaatcgtttataacgtaaaccaagatactttagacagctctgacgtaatcgtttcag | 420 | |
| 421 | gtgcttcttgtactacaaactctcttgctcctgtagcacaagttttaaatgacagctttg | 480 | |
| 481 | gtttagttgaaggtttcatgactactatccatgcttatactggtgaccaaaatactcaag | 540 | |
| 541 | atggtccacacagaaaaggtgacaaacgtcgtgctcgtgcagcagctgaaaacatcgttc | 600 | |
| 601 | caaactcaactggtgcagctaaagcaattggtaaagtaattcctgaaatcgacggaaaat | 660 | |
| 661 | tagacagcggcgctcaacgtgttcctgttgcaactggttcattagctgaattaacagttg | 720 | |
| 721 | tattagataaagacgttacagttgacgaagtaaacgaagcaatgaaacaagcttctaacg | 780 | |
| 781 | aatcattcggttacaacgaagacgaaatcgtatcttcagatgttgtaggtatgacattcg | 840 | |
| 841 | gttcattatttgatgcaactcaaactcgtgtaatgactgtttcaggccgtcaattagtta | 900 | |
| 901 | aagtagcagcttggtatgataacgaaatgtc | | |

(6) Staphylococcus chromogenes

| | | | |
|---|---|---|---|
| 1 | atggttttggtagaattggtcgtttagcattcagaagaattcaagacgtagaaaatattg | 60 | (SEQ ID NO:6) |
| 61 | aggttgtagctgtaaacgatttaacagacgacgatatgcttgcacatttattgaaatatg | 120 | |
| 121 | acacaatgcaaggtcgttttactgaagaagtagatgtaattgatggtggtttccgcgtaa | 180 | |
| 181 | atggtaaagaagtgaaatcattctctgaaccagaaccatcaaaattaccatggaaagatc | 240 | |
| 241 | ttgacgtagatgttgttttagaatgtacaggtttctttacatcaaaagaaaaagcagaag | 300 | |
| 301 | ctcacattgaagcaggtgctaaaaaagtattaatttctgcaccaggaactggcgatctta | 360 | |
| 361 | aaacaatcgtatataatgtcaaccatgaagaattagacggttctgaaacagttgtatcag | 420 | |
| 421 | gtgcttcttgtacaacaaactcattagcaccagtagcaaaaactttaaatgatgaatttg | 480 | |
| 481 | gtatcgttgaaggtttaatgactacaattcacgcatacactggtgaccaaaatacacaag | 540 | |
| 541 | actcaccacacagaaaaggtgacaaacgtcgtgcacgtgcagctgcagaaaacattattc | 600 | |
| 601 | ctaactcaacaggtgctgcgaaagcaatcggtttagttatccctgaaattgatggaaaat | 660 | |
| 661 | tagacggtggcgcacaacgtgtaccagtagcaacaggttcattaactgaattaacagttg | 720 | |
| 721 | ttttagataaagaagtatcagtagaagacgttaacaatgcaatgaaaaatgcaacaaacg | 780 | |
| 781 | aatcattcggttacactgaagacgaaattgtatcttcagatgttgtaggcatgactttcg | 840 | |
| 841 | gagcattatttgatgcaactcaaacacgtgtaatgactgttggcgaccgtcaattagtta | 900 | |
| 901 | aagtagcagcttggtatgataacgaaatgtc | | |

(7) Staphylococcus cohni

| | | | |
|---|---|---|---|
| 1 | atggttttggtagaattggtcgtttagcatttagaagaattcaagatgtagaaggaatcg | 60 | (SEQ ID NO:7) |
| 61 | atgtagtagcagtaaacgacttaacagatgatgaaatgttagctcatttattaaaatatg | 120 | |
| 121 | acactacacaaggtcgcttcacaggagaagttgaagttgaagaaaatggtttccgcgtta | 180 | |
| 181 | acggattagaagttaaatcattttctgagccagatccaagtaaattaccttggggagact | 240 | |
| 241 | tagatatcgatgttgtattagaatgtactggtttattcacagataaagaaaaagcagaag | 300 | |
| 301 | cacacatcaatgcaggcgctaaaaaagtattaatttcagcaccagctaaaggtgacttaa | 360 | |
| 361 | aaacaatcgtatacaacactaaccacgacactttagatggttcagaagatgttgtttcag | 420 | |
| 421 | gtgcttcatgtactactaactcattagcaccagttgctaaagttttaaatggtgaattcg | 480 | |
| 481 | gcttaatcgaaggtttcatgactacaatccacgcatatactggtgaccaaagcacacaag | 540 | |
| 541 | atgcgcctcacagaaaaggtgacaaacgtcgtgcgcgtgcagctgctgaaaacattatcc | 600 | |
| 601 | ctaactcaacaggtgctgctaaagctatcggcctagtaattcctgaaattgatggtaaat | 660 | |
| 661 | tagacggtggagcacaacgtgtaccagttgcaactggttcattaactgaattaacagtag | 720 | |
| 721 | tattagataaaaatgtaagtatcgaagacgtaaataacgcaatgaaaaatgcatctaacg | 780 | |
| 781 | aatcattcggttatactgaagacgaaatcgtttcttctgacgtaatcggaatgacatacg | 840 | |
| 841 | gttcattatttgatgcaactcaaacacgtgttatgactgttggagatcgtcaattagtta | 900 | |
| 901 | aagtagcagcttggtatgataacgaaatgtc | | |

(8) Staphylococcus delphini

| | | | |
|---|---|---|---|
| 1 | atggttttggtagaattggtcgtttagcatttagaagaattcaagatgtggaaaatatcg | 60 | (SEQ ID NO:8) |
| 61 | acgttgtagcagtaaacgatttaacagacgacgatatgcttgcacacttattaaaatatg | 120 | |
| 121 | actcaacacaaggtcgttttactgaagaagtagaagtaattgacggtggtttccgtgtaa | 180 | |
| 181 | atggtaaagaagtgaaatcattctctgaaccagaacctaaaaacttaccatggggcgagt | 240 | |
| 241 | tagacatcgacgtggtattagaatgtactggtttcttcactgataaagaaaaagctgaag | 300 | |
| 301 | cacacatcgaagcaggcgcgaaaaaagtattaatttctgcaccagctaaaggtgacctta | 360 | |
| 361 | aaacagttgtatataacgttaaccacgaaattttagatggtactgaaacagttgtttctg | 420 | |
| 421 | gtgcttcatgtacaacaaactcattagcacctgttgcaaaaactttacaagacaacttcg | 480 | |
| 481 | gtatcgttgaaggtttaatgacaacaattcacgcttacactggtgaccaaaacacattag | 540 | |
| 541 | acgcacctcacagaaaaggtgacaaacgtcgtgcgcgtgcagctgctgaaaacatcatcc | 600 | |
| 601 | ctaactcaactggtgctgcgaaagcaatcggtttagttattcctgaaatcgatggtaaat | 660 | |
| 661 | tagacggtggtgcacaacgtgttcctgtagcaacaggttcattaactgaattaactgtag | 720 | |
| 721 | tattagaaaaagaagtctctgttgaagaagttaacaaagtcatgaaagaggcaactaacg | 780 | |
| 781 | aatcattcggttacactgaagacgaaatcgtttcttcagacgttgttggtatgactttcg | 840 | |
| 841 | gtgctttattcgacgcaactcaaacacgtgtaatgactgttggcgaccgtcaattagtta | 900 | |
| 901 | aagttgcagcttggtatgataacgaaatgtc | | |

(9) Staphylococcus epidermidis

| | | | |
|---|---|---|---|
| 1 | atggttttggtagaattggtcgtttagcatttagaagaattcaagacgtagaaggtttag | 60 | (SEQ ID NO:9) |
| 61 | aagtggttgcagtaaacgacttaactgatgacgatatgttagcacacttattaaaatatg | 120 | |
| 121 | acactatgcaaggtcgcttcactggtgaagtagaagttatcgacggtggattccgtgtta | 180 | |
| 181 | acggtaaagaagttaaatcattcgatgaaccagatgcaagcaaattaccttggaaagact | 240 | |
| 241 | tagatatcgacgtagtattagaatgtactggtttctatactgacaaagataaagcacaag | 300 | |
| 301 | ctcatgttgacgcaggtgctaaaaaagtattaatctcagctccagcaactggtgacttaa | 360 | |
| 361 | aaacaatcgtttacaacactaaccacgatgaattagatggttctgaaacagtagtatcag | 420 | |
| 421 | gtgcatcatgtactactaactcattagctcctgtagctaaagtgttaagtgatgagttcg | 480 | |
| 431 | gtttagttgaaggtttaatgacaactatccacgcttacactggtgaccaaaatactcaag | 540 | |
| 541 | atgctccacacagaagaggcgacaaacgtcgtgctcgtgctgcagcagaaaacatcatcc | 600 | |
| 601 | ctaactcaactggtgctgctaaagcaatcggtaaagtaattcctgaaatcgacggaaaat | 660 | |
| 661 | tagacggtggtgcacaacgtgttccagttgcaactggttcattaactgaattaacagttg | 720 | |
| 721 | tattagaaaaagacgtaacagttgaacaagttaacgaagcaatgaaacaagcttctgacg | 780 | |
| 781 | aatcattcggttacactgaagacgaaatcgtatcttctgatgtagttggtatgacttacg | 840 | |
| 841 | gttcattattcgatgcaactcaaactcgtgtaatggctgttggtggtcgtcaattagtta | 900 | |
| 901 | aagtagcagcttggtatgataacgaaatgtc | | |

(10) Staphylococcus equorum

| | | | |
|---|---|---|---|
| 1 | atggttttggtagaattggtcgtttagcatttagaagaattcaagacgtagaaggtattg | 60 | (SEQ ID NO:10) |
| 61 | acgtagtagcagttaacgatttaacagatgacgaaatgttagctcatttattaaaatatg | 120 | |
| 121 | acactacacaaggtcgcttcacaggagaagttgaagtagaaaaagacggattccgtgtaa | 180 | |
| 181 | atggacaagaagttaaatcattctcagaacctgaaccaagtaaattaccttggaaagatt | 240 | |
| 241 | tagacatcgatgttgttttagaatgtactggtttcttcgctgataaagaaaaagcagaag | 300 | |
| 301 | ctcacattgacgctggcgctaaaaaagtattaatctctgcaccagcaacaggcgacttaa | 360 | |
| 361 | aaacaatcgtttataacactaaccacagtgaattagatggttcagaaacagttgtttcag | 420 | |
| 421 | gtgcttcatgtactactaactcattagctccagtagctaaagttttaaatgacgacttcg | 480 | |
| 481 | gcttagttgaaggtttcatgactactattcacgcatatactggtgaccaaagtactcaag | 540 | |
| 541 | atgctccacacagaaaaggcgacaaacgccgtgcacgtgcagctgctgaaaacatcatcc | 600 | |
| 601 | ctaactcaacaggtgctgctaaagcaattggtttagtaatccctgaaatcgatggtaaat | 660 | |
| 661 | tagacggtggcgctcaacgtgttccagttgctactggttcattaactgaacttacagttg | 720 | |
| 721 | tattagaaaaagacgtaagcgttgaagacgttaacgcagcaatgaaaaatgcttcagacg | 780 | |
| 781 | aatcatttggttacactgaagacgaaatcgtttcttctgacgtaatcggtatgacttacg | 840 | |
| 841 | gttcattattcgatgcaacgcaaactcgtgttatgacagttggagatcaccaattagtta | 900 | |
| 901 | aaatagcagcttggtatgataacgaaatgtc | | |

(11) Staphylococcus gallinarum

| | | | |
|---|---|---|---|
| 1 | atggttttggtagaattggtcgtttagcattcagaagaattcaaaacgttgaaggaatcg | 60 | (SEQ ID NO:11) |
| 61 | acgtagtagcagtaaatgacttaacagatgacgaaatgttagctcacttattaaaatatg | 120 | |
| 121 | atactatgcaaggtcgcttcactggagaagttgaagttgaaaaagacggtttccgtgtta | 180 | |
| 181 | acggtcaagaagttaaatcattctctgagccagacccaagtaaattaccatggggtgact | 240 | |
| 241 | tagacatcgatgtagtattagaatgtactggtttcttcgctgacaaaactaaagcagaag | 300 | |
| 301 | ctcacatcaatgcaggtgctaaaaaagtattaatctcagctccagcaactggtgacttga | 360 | |
| 361 | aaacaatcgttttcaacactaaccataacgaattagatggtacagaaacagttgtttcag | 420 | |
| 421 | gtgcttcatgtactactaactcattagctccagtagctaaagtattaaatgatgactttg | 480 | |
| 481 | gtttagttgaaggtcttatgactacaattcacgcttacactggtgaccaaaatacacaag | 540 | |
| 541 | atgctccacatgctaaaggtgacaaacgccgtgctcgtgcagctgctgaaaatatcatcc | 600 | |
| 601 | ctaactcaactggtgctgctaaagcaatcggtaaagttatccctgaaattgacggcaaat | 660 | |
| 661 | tagacggtggtgcgcaacgtgtaccagttgctactggttctttaactgaattaacagttg | 720 | |
| 721 | tattagaaaaagacgtaagcgttgaagacgttaacaatgcaatgaaaaacgcttcaaacg | 780 | |
| 781 | aatcattcggttacactgaagacgaaatcgtatcttctgacgtagttggtatcacttacg | 840 | |
| 841 | gtccattattcgatgcaacacaaactcgtgtaatgactgttggcgatcgtcaattagtta | 900 | |
| 901 | aagttgcagcttggtatgataacgaaatgtc | | |

(12) Staphylococcus haemolyticus

| | | | |
|---|---|---|---|
| 1 | atggttttggtagaattggtcgtttagcatttagaagaattcaagacgtaggaggtattg | 60 | (SEQ ID NO:12) |
| 61 | aagtagttgcagtaaacgacttaacagacgacgaaatgttagctcatttattaaaatatg | 120 | |
| 121 | acactatgcaaggtcgtttcacaggagaagttgaagttgttgatggtggtttccgcgtaa | 180 | |
| 181 | atggtaaagaagttaaatcatacgaagaaccagatgcaagcaaattaccttggggcgatt | 240 | |
| 241 | tagatatcgacgtagtattagaatgtactggtttctatacagataaagaaaaagcagaag | 300 | |
| 301 | cacacatcaatgcaggtgctaaaaaagtattaatctctgcaccagctcaaggtgatgtaa | 360 | |
| 361 | aaacaatcgtattcaacactaaccacaatgacttagatggttcagaaacagttgtttcag | 420 | |
| 421 | gtgcatcatgtactactaactcattagcaccagttgctaaagtattaagtgatgaatttg | 480 | |
| 481 | gtttagttgaaggtttaatgacaactattcacgcatacactggtgaccaaatgactcaag | 540 | |
| 541 | acggtccacataaaaaaggtgacaaacgtcgtgcacgtgcagcagctcaaaacatcgtac | 600 | |
| 601 | ctaactcaacaggtgctgcaaaagcaatcggtaaagttattcctgaaatcgatggtaaat | 660 | |
| 661 | tagacggtggtgctcaacgtgtaccagttgctacaggttcattaactgaagtgacagttg | 720 | |
| 721 | tattagaaaaagacgttactgttgaagacgttaacaaagcaatgaaaaacgcttcaaacg | 780 | |
| 781 | aatcatttggttacactgaagacgaaatcgtttcttctgacgtagttggcatgacttacg | 840 | |
| 841 | gttcattattcgatgctactcaaactcgtgtaatgtctgttggtgaccgtcaattagtta | 900 | |
| 901 | aagttgcagcttggtatgataacgaaatgtc | | |

(13) Staphylococcus hominis

| | | | |
|---|---|---|---|
| 1 | atggttttggtagaattggtcgtttagcattcagaagaattcaagacgtagaaggtattg | 60 | (SEQ ID NO:13) |
| 61 | aagtagttgcagtaaacgacttaacagacgacgaaatgttagctcatttattaaaatatg | 120 | |
| 121 | acactatgcaaggtcgctttaacggagacgtagaagtagttgaaggtggtttccgtgtaa | 180 | |
| 181 | atggtaaagaagttaaatcttttgaagaaccagatgcaagtaaattaccatggggcgatt | 240 | |
| 241 | tagatatcgacgtagtattagaatgtactggtttctatacagaaaaagaaaaagctgaag | 300 | |
| 301 | cacacattaatgcaggagctaaaaaagtattaatttctgctccagccaaaggtgatgtta | 360 | |
| 361 | aaactatcgtatttaacacaaaccacaaagacttagatggatctgaaacagtagtatcag | 420 | |
| 421 | gtgcttcatgtactacaaactcattagcaccagttgctaaagttttaaatgacgaatttg | 480 | |
| 481 | gtattgttgaaggtttaatgacaactatccatgcttacactggtgaccaaatgactcaag | 540 | |
| 541 | atggtccacacagaaaaggtgacaaacgtcgtgcacgtgcagcagcacaaaacatcgtac | 600 | |
| 601 | ctaactcaacaggtgcagctaaagctatcggtaaagtaattcctgaaatcgatggtaaat | 660 | |
| 661 | tagatggtggagcacaacgtgtaccagtagctactggttcattaactgaagtaacagttg | 720 | |
| 721 | tattagaaaaagaagtaacagttgaagatgttaacaaagcaatgaaaaatgctgctgacg | 780 | |
| 781 | aatcattcggttacactgaagatgaaatcgtatcatcagatgttgctggtatgaactttg | 840 | |
| 841 | gttcattattcgatgcaactcaaactcgtgtcatctcagttggcgacaaacaattagtta | 900 | |
| 901 | aagtagcagcttggtatgataacgaaatgtc | | |

(14) Staphylococcus hyricus

| | | | |
|---|---|---|---|
| 1 | atggttttggtagaattggtcgtttagcatttagaagaattcaagacgtagaaaacattg | 60 | (SEQ ID NO:14) |
| 61 | aggtagtagctgtcaatgatttaactgacgacgacatgcttgcacatttattaaaatatg | 120 | |
| 121 | acacgatgcaaggacgttttactgaagaagtagatgtaattgatggtggtttccgcgtaa | 180 | |
| 181 | atggtaaagaagtgaaatcattctctgaaccagaaccatctaaattaccttggaaagact | 240 | |
| 241 | tagaagtagatgttgttttagaatgtactggtttcttcacatctaaagaaaaagctgaag | 300 | |
| 301 | cacacattgaagcaggcgctaaaaaagtcttaatttcagcaccaggtactggtgatctta | 360 | |
| 361 | aaacaattgtatataacgttaaccatgaagaattagacggttcagaaacagttgtttcag | 420 | |
| 421 | gtgcgtcttgtactacaaactcattagctccggtagcgaaaacattacacgatgaatttg | 480 | |
| 481 | gtattgttgaaggtttaatgactacaattcacgcttatacaggtgaccaaaatacgcaag | 540 | |
| 541 | actcacctcacagaaaaggtgacaaacgtcgtgcacgtgcagctgctgaaaacatcatcc | 600 | |
| 601 | ctaactcaacaggtgctgcaaaagcaatcggtttagttattccagaaattgctggtaaat | 660 | |
| 661 | tagatggtggcgcgcaacgtgtaccagttgctacaggttcattaacagaattaactgttg | 720 | |
| 721 | ttttagaaaaagaagtatctgttgaagaagttaacaatgcaatgaaaaatgcaactaatg | 780 | |
| 781 | aatcattcggttacactgaagatgaaatcgtctcttctgacgttgtaggtatgacgtttg | 840 | |
| 841 | gtgcattattcgacgcaactcaaacacgcgttatgactgttggcgatcgtcaattagtta | 900 | |
| 901 | aagttgcagcttggtatgataacgaaatgtc | | |

(15) Staphylococcus intermedius

| | | | |
|---|---|---|---|
| 1 | atggttttggtagaattggtcgtttagcattccgtcgtattcaaaatgtggaaggaattg | 60 | (SEQ ID NO:15) |
| 61 | aagttgttgcaatcaatgacttaacagacgctaaaatgttagctcatctgttaaaatatg | 120 | |
| 121 | atacaactcaaggccgttttgacggtgaagtagaagtacatgatggtttcttcaaagtaa | 180 | |
| 181 | acggtaaagaagttaaagtattagctaaccgtaacccagaagaacttccatggggtgaac | 240 | |
| 241 | taggagtagacatcgttcttgaatgtactggtttcttcacagcacaagacaaagctgaat | 300 | |
| 301 | tacacattaaagctggcgctaaaaaagttgttatctccgctccagcaactggcgacatga | 360 | |
| 361 | aaacaatcgtttacaatgtaaaccatgaaacattagacggaactgaaacagttatttctg | 420 | |
| 421 | gtgcaagctgtactactaactgtttagctccaatggctaaagttttagaagacaaatttg | 480 | |
| 481 | gtgttgttgaaggcctaatgactacaattcacgcatacactggtgaccaaaatacattag | 540 | |
| 541 | acgctccacatccaaaaggtgacttccgtcgtgctcgtgctgctgcagaaaatatcatcc | 600 | |
| 601 | ctaatacaactggtgctgcaaaagctatcggtgaagtattaccaagccttaaaggtaaat | 660 | |
| 661 | tagacggagcagctcaacgtgttccagttccaactggttcccttactgaattagtaacag | 720 | |
| 721 | ttcttaacaaaaaagttactgttgatgaagtaaatgcagctatggaagcagcttctgatc | 780 | |
| 781 | cagaaacattcggttacactaatgacgcaatcgtttcttctgatatcaaaggtatgactt | 840 | |
| 841 | tcggttctttatttgacgaaactcaaacaaaagttcttacagttggcgatcaacaattag | 900 | |
| 901 | ttaaaactgcagcttggtatgataacgaaatgtc | | |

(16) Staphylococcus kloosii

| | | | |
|---|---|---|---|
| 1 | atggttttggtagaattggtcgtttagcatttagaagaattcaaaacgttgacggaatcg | 60 | (SEQ ID NO:16) |
| 61 | atgtagtagcagttaacgacttaacagatgacgaaatgttagcacacttattaaaatatg | 120 | |
| 121 | acacaatgcaaggtcgtttcactggagaagttgaagttgaagaaaacggcttccgcgtaa | 180 | |
| 181 | atggtcaagaagttaaatcattctctgaaccagatccaagtaaattaccatggggcgact | 240 | |
| 241 | tagatatcgatgttgtcttagaatgtactggtttatttgctgataaagataaagcttcag | 300 | |
| 301 | ctcatatcgatgcaggcgctaaaaaagttttaatttcagctccagctacaggcgacttaa | 360 | |
| 361 | aaacaatcgtttacaacactaaccacaacgaattagacggttcagaatcagtagtatcag | 420 | |
| 421 | gtgcttcatgtactactaactcattagctccagtagctaaagttttaaatgatgaattta | 480 | |
| 481 | gtttagttgaaggtttaatgacaactatccacgcttacactggtgaccaaagcacacaag | 540 | |
| 541 | atgctcctcacagaaaaggtgacaaacgtcgtgctcgtgcagcagcagaaaacatcatcc | 600 | |
| 601 | ctaactcaacaggtgctgcaaaagcaatcggtttagttattcctgaaatcgacggaaaat | 660 | |
| 661 | tagacggtggcgcacaacgtgttccagttgcaacaggttcattaactgaattaacagttg | 720 | |
| 721 | tattagaaaaagacgtaagtgttgaagatgtaaacaacgcaatgaaaaatgcttcaaacg | 780 | |
| 781 | aatcatttggttacactgaagacgaaatcgtttcttctgacgtaatcggtatgacttacg | 840 | |
| 841 | gttcattattcgacgctacacaaactcgtgtaatgactgttggtgactcgtcaattagtt | 900 | |
| 901 | aaagttgcagcttggtatgataacgaaatgtc | | |

(17) Staphylococcus lentus

| | | | |
|---|---|---|---|
| 1 | atggttttggtagaattggtcgtttagcatttagaagattacaagaagtagaaaatatcg | 60 | (SEQ ID NO:17) |
| 61 | aagtagtagcaatcaacgatttagcagatgacgctatgttagctcatttattaaaatacg | 120 | |
| 121 | attctacacaaggtcgtttcaaagatgaagtagaagtaattgaaggcggattccgtgtaa | 180 | |
| 181 | acggtaaagaaatcaaaactttcgaaaatcctaaccctaaagaattaccctggggagact | 240 | |
| 241 | tagacatcgatgtagtattagaatgtactggtttcttcgctgataaagaaaaagctcatg | 300 | |
| 301 | ctcacatcgccgcaggtgctaaaaaagtattaatttcagctccagcttcaggcgacttga | 360 | |
| 361 | aaacaatcgtatacaatgttaaccatgatgaattagacggttcagaagaaatcgtatctg | 420 | |
| 421 | gtgcatcttgtactactaactgtttagctccaatggctaaagtattaaatgatgaattcg | 480 | |
| 481 | gtatcgttgaaggattaatgatgacaattcatgcttatactggtgaccaaaatacactag | 540 | |
| 541 | atgctccacatgctaaaggtggcttccgtcgtgctcgtgcagcagctgaaaacatcgtac | 600 | |
| 601 | ctaactcaactggtgcagctaaagcaattggcttagttatcccagaattaaaaggtaaat | 660 | |
| 661 | tagatggatcagctcaacgtgttccagtagcaactggttcagtaactgaattaacagcag | 720 | |
| 721 | tattagataaagaagtatctatagaagaaatcaacgaagcaatgaaaaatgctacaaatg | 780 | |
| 781 | attcattcggatacactgaagacgaaatcgtttcttctgatgttattggcatcacttacg | 840 | |
| 841 | gttcattattcgacgcaactcaaactcgtgtaatgacagttggagaccgtcaattagtta | 900 | |
| 901 | aaactgcagcttggtatgataacgaaatgtc | | |

(18) Staphylococcus lugdunesis

| | | | |
|---|---|---|---|
| 1 | atggttttggtagaattggtcgtttagcatttagaagaattcaagacgtagaaggcatcg | 60 | (SEQ ID NO:18) |
| 61 | aggtagtagcagtaaacgacttaacagatgatgatatgttagcgcatttattaaaatatg | 120 | |
| 121 | acactatgcaaggtcgcttcactagcgaagttgaagttgttgatggtggtttccgtgtaa | 180 | |
| 181 | atggtaaagaagttaaatcatttgaagaaccagacgcaagcaaattaccatggggtgacc | 240 | |
| 241 | taggtgttgacgtggtattagaatgtactggattctatacagataaagaaaaagctgaag | 300 | |
| 301 | cacacattcatgcaggtgctaaaaaagtattaatttctgcgccagctaaaggtgacgtta | 360 | |
| 361 | aaactatcgtttacaacactaaccatagtgacttagacggttcagaaacagttgtatcag | 420 | |
| 421 | gtgcttcatgtacaactaactctttagcaccagtagctaaagtaatcagcgatgaatttg | 480 | |
| 481 | gtttagtagaaggtttaatgacaactattcatgcatacactggtgaccaaatgactcaag | 540 | |
| 541 | atggtccacacagaaaaggtgacaaacgtcgtgctcgtgcagctgcacaaaacatcgtac | 600 | |
| 601 | ctaactcaactggtgctgctaaagcaatcggtaaagttattcctgaaatcgatggtaaat | 660 | |
| 661 | tagacggtggtgcgcaacgtgtacctgtagctacaggttcattaactgaattaactgtag | 720 | |
| 721 | tattagaaaaacaagatgtaacagtagaacaagtaaacgaagcgatgaaaaaagcttcta | 780 | |
| 781 | acgaatcattcggttacaatgaagatgaaattgtttcttctgacgtagttggtatgactt | 840 | |
| 841 | acggttcattatttgatgcaacacaaactcgtgtaatgtcagttggtggccgtcaattag | 900 | |
| 901 | ttaaagttgcagcttggtatgataacgaaatgtc | | |

(19) Staphylococcus piscifermentans

| | | | |
|---|---|---|---|
| 1 | atggttttggtagaattggtcgtttagcattcagaagaattcaagatgttgaaggtatcg | 60 | (SEQ ID NO:19) |
| 61 | atgtagtagcagtaaacgacttaacagatgatgaaatgttagcgcatttattaaaatacg | 120 | |
| 121 | atacaatgcaaggacgtttcacagaagaagttgaagttgtagatggcggattccgtgtga | 180 | |
| 181 | atggtaaagaagttaaatcattcgaagaaccagatgcaagcaaattaccttggaaagatt | 240 | |
| 241 | tagacattgatgttgtattagaatgtactggtttctatacaagtgatgaaaaagctaaag | 300 | |
| 301 | cacatatcgacgcaggtgctaaaaaagtattaatttctgctccagcaactggcgatgtta | 360 | |
| 361 | aaacaatcgtttataacgtaaaccaagatactttagacagctctgatgttatcgtttcag | 420 | |
| 421 | gtgcttcttgtactacaaactcacttgctccagtagcaaaagttttaaacgacagctttg | 480 | |
| 481 | gtttagttgaaggtttcatgactactattcatgcttacactggtgaccaaaatactcaag | 540 | |
| 541 | atggtccacacagaaaaggtgacaaacgtcgtgcacgtgcagcagctcaaaacatcgtac | 600 | |
| 601 | caaactcaactggtgctgctaaagcaatcggtaaagtaatccctgaaattgacggtaaat | 660 | |
| 661 | tagacggtggtgctcaacgtgttcctgttgcaactggttcattaacagaattaacagttg | 720 | |
| 721 | tattagacaaagaagtttcagttgacgaagtaaacgaagcaatgaaacaagcttctaacg | 780 | |
| 781 | aatcattcggttacaatgaagacgaaatcgtatcttctgacgtggttggtatgacattcg | 840 | |
| 841 | gttcattattcgatgctacacaaactcgtgtgatgactgtatcaggtcgtcaattagtta | 900 | |
| 901 | aagttgcagcttggtatgataacgaaatgtc | | |

(20) Staphylococcus saprophyticus

| | | | |
|---|---|---|---|
| 1 | atggttttggtagaattggtcgtttagcattcagaagaattcaaaacgttgacggaatcg | 60 | (SEQ ID NO:20) |
| 61 | acgtagtagcagtaaacgatttaacagatgacgaaatgttagctcatttattaaaatatg | 120 | |
| 121 | atactatgcaaggacgcttcacaggagaagttgaagtagaaaaagacggtttccgcgtaa | 180 | |
| 181 | acggacaagaagtaaaatcattctctgagcctgaaccaagtaaattaccttggaaagact | 240 | |
| 241 | tagacatcgatgttgtattagaatgtactggtttcttcgctgataaagaaaaagcagaag | 300 | |
| 301 | cacacatcaatgcaggtgctaaaaaagtattaatctctgctccagctacaggcgatttaa | 360 | |
| 361 | aaacaatcgtttataatacaaaccaccaagaattagacggttcagaaactgttgtttcag | 420 | |
| 421 | gtgcttcatgtactactaactcattagctcctgttgctaaagttttaaatgatgacttcg | 480 | |
| 481 | gtttagtagaaggtttcatgactactatccacgcatacactggtgaccaaagcacactag | 540 | |
| 541 | atgcaccacacagaaaaggcgacaaacgtcgtgcgcgtgcagctgctgaaaacatcatcc | 600 | |
| 601 | ctaactcaactggtgctgctaaagcaattggcttagtaattcctgaaattgatggtaaat | 660 | |
| 661 | tagatggaggagcgcaacgtgttcctgttgcaactggttcattaactgaattaacagttg | 720 | |
| 721 | ttttagnnnnnnntgtaagcattgaagatgtaaatgctgcaatgaaaaatgcttcaaacg | 780 | |
| 781 | aatcattcggttacacagaagacgaaatcgtatcttcagacgtaatcggtatgacttatg | 840 | |
| 841 | gttcattatttgatgcaacacaaactcgtgtaatgactgttggcgaccgtcaattagtta | 900 | |
| 901 | aagtagcagcttggtatgataacgaaatgtc | | |

(21) Staphylococcus schleiferi

| | | | |
|---|---|---|---|
| 1 | atggttttggtagaattggtcgtttagcatttagaagaattcaagatgtagaaaacattg | 60 | (SEQ ID NO:21) |
| 61 | aggtcgtagctgtaaacgatttaacagatgacgatatgcttgcacatttattgaaatatg | 120 | |
| 121 | acacaatgcaaggacgttttactgaagaagtggaagtaattgatggtggtttccgtgtga | 180 | |
| 181 | atggtaaagaagcgaaatcattctctgaaccagaacctgctaaattaccttggggtgacc | 240 | |
| 241 | ttggtgtggacgtagtattagaatgtactggtttcttcacagataaagaaaaagctgaag | 300 | |
| 301 | cacacattcaagcaggcgctaaaaaagtattaatctcagcaccggctaaaggtgatctta | 360 | |
| 361 | aaacaatcgtatataatgttaaccacgacgatttagatggttctgaaacagttgtttcag | 420 | |
| 421 | gtgcatcatgtactacaaactcattagcacctgttgcaaaaactttacacgacgaattcg | 480 | |
| 481 | gtattgttgaaggtttaatgactacaattcacgcatatactggtgaccaaaatacacaag | 540 | |
| 541 | atgcacctcacagaaaaggtgacaaacgtcgtgcgcgtgctgctgctgaaaacattatcc | 600 | |
| 601 | ctaactctacaggggcagcaaaagcaatcggtttagttattccagaaattgccggtaaat | 660 | |
| 661 | tagacggtggtgcacaacgtgttccagttgcaactggttcattaacagaattaacagttg | 720 | |
| 721 | ttttagataaagaagtgactgttgaagaagtaaacaaagtattgaaagcagcaactaacg | 780 | |
| 781 | aaccattcggttacactgaagacgaaattgtttcttcagacgttgtaggtatgacttacg | 840 | |
| 841 | gtgcattattcgatgcaactcaaactcgtgtaatgactgttggcgaccgtcaattagtta | 900 | |
| 901 | aagttgcagcttggtatgataacgaaatgtc | | |

(22) Staphylococcus sciuri

| | | | |
|---|---|---|---|
| 1 | atggttttggtagaattggtcgtttagcatttagaagattacaagaagttgaaaatatcg | 60 | (SEQ ID NO:22) |
| 61 | aagtagtagcaatcaacgatttaacagatgacgcaatgttagctcatttattaaaatatg | 120 | |
| 121 | attcaacacaaggtcgtttcaaagacgaagtagaagttatcgaaggcggattccgcgtaa | 180 | |
| 181 | acggtagagaaatcaaaactttcgaaaatcctaatcctaaagaattaccatggggcgatt | 240 | |
| 241 | tagatatcgatgtagtattagaatgtactggtttcttcgctgacaaagacaaagcttcag | 300 | |
| 301 | ctcacatcgaagcaggtgctaaaaaagtattaatttcagctccagcatcaggtgacttaa | 360 | |
| 361 | aaactatcgtttataacgttaaccatgacgaattagacggatctgaagaaatcgtttcag | 420 | |
| 421 | gtgcatcttgtacaactaactgtttagctccaatggctaaagtattaaatgatgaattcg | 480 | |
| 481 | gtatcgttgaaggtttaatgatgacaattcacgcttacactggtgaccaaaatactttag | 540 | |
| 541 | atgctccacatgctaaaggtgacttccgtcgtgctcgtgcagctgctcaaaacatcgtgc | 600 | |
| 601 | ctaactcaactggtgctgctaaagcaatcggtttagtaattccagaattaaaaggtaaat | 660 | |
| 661 | tagatggatcagctcaacgtgttccagtagcaactggttcagtaacagaattaacagctg | 720 | |
| 721 | tattggacaaagaagtttcagttgaagaaatcaatgcagcaatgaaaaatgctacaaatg | 780 | |
| 781 | attcattcggttacactgaagacgaaatcgtatcttctgatatcattggtatcacttacg | 840 | |
| 841 | gttcattatttgatgcaactcaaactcgtgttatgacagttggagatcgccaattagtta | 900 | |
| 901 | aaactgcagcttggtatgataacgaaatgtc | | |

(23) Staphylococcus simulans

| | | | |
|---|---|---|---|
| 1 | atggttttggtagaattggtcgtttagcatttcagaagaattcaagatgttgaaggtatc | 60 | (SEQ ID NO:23) |
| 61 | gatgtagtagcagtaaacgacttaacagatgatgaaatgttagcacacttattaaaatac | 120 | |
| 121 | gatacaatgcaaggacgtttcactgaagaagttgaagttgtagatggcggattccgcgtg | 180 | |
| 181 | aatggtaaagaagttaaatcattcgaagaaccagatgcaagcaaattaccttggaaagat | 240 | |
| 241 | ttagacattgatgtcgtattagaatgtactggtttctacactagcgacgaaaaagcacaa | 300 | |
| 301 | gctcacattgacgcaggtgctaaaaaagtattaatctctgcaccagcaactggtgacgtt | 360 | |
| 361 | aaaacaatcgtttataacgtaaaccaagatactttagacagctctgacgtaatcgtttca | 420 | |
| 421 | ggtgcttcttgtactacaaactcacttgctccagtagcaaaagtattaaatgacagcttc | 480 | |
| 481 | ggtttagtagaaggtttcatgactactatccacgcttacactggtgaccaaaatactcaa | 540 | |
| 541 | gacggtccacacagaaaaggcgacaaacgtcgtgcacgtgcagcagctgaaaacatcgtt | 600 | |
| 601 | cctaactcaactggtgctgctaaagcaatcggtaaagtaattcctgaaatcgacggaaaa | 660 | |
| 661 | ttagacggtggcgctcaacgtgttcctgtagcaactggttcattaactgaattaacagtt | 720 | |
| 721 | gtattagacaaagacgtaacaatcgaagaagtaaacgaagctatggaagcagcttctaac | 780 | |
| 781 | gaatcattcggttacaacgaagacgaaatcgtatcttcagacgtagttggtatgacattc | 840 | |
| 841 | ggttcattattcgatgcaactcaaactcgtgttatgactgtatctggtcgtcaattagtt | 900 | |
| 901 | aaagttgcagcttggtatgataacgaaatgtc | | |

(24) Staphylococcus xylosus

| | | | |
|---|---|---|---|
| 1 | atggttttggtagaattggtcgtttagcatttagaagaattcaaaacgttgacggaattg | 60 | (SEQ ID NO:24) |
| 61 | acgtagtagcagtaaatgacttaacagatgacgaaatgttagcacatttattaaaatatg | 120 | |
| 121 | acactatgcaaggacgcttcacaggagaagttgaagtagaaaatgacggtttccgtgtaa | 180 | |
| 181 | acggacaagaagtaaaatcattctctgagccagacccaagcaaattaccttggaaagatt | 240 | |
| 241 | tagacatcgatgttgtattagaatgtactggtttctacgctgataaagaaaaagcagaag | 300 | |
| 301 | ctcacattaatgcaggtgctaaaaaagtattaatttcagctccagctactggtgatttaa | 360 | |
| 361 | aaacaatcgtttataacacaaaccaccaagagttagatggtaaagaaacagttgtttcag | 420 | |
| 421 | gtgcttcatgtacaactaactcattagctccagttgctaaagtattaaatgatgactttg | 480 | |
| 481 | gtttagtagaaggtttcatgactacaattcatgcttacactggtgaccaaaatacacaag | 540 | |
| 541 | atgcgccacacgctaaaggcgacaaacgtcgtgctcgtgcagctgctgaaaacattatcc | 600 | |
| 601 | ctaactcaactggtgctgctaaagcaattggcctagttatccctgaaattgatggtaaat | 660 | |
| 661 | tagacggtggagcgcaacgtgttcctgtagctactggttcgttaacagaattaacagttg | 720 | |
| 721 | tattagacaaaaatgtaagtgttgaagacgttaatgctgcaatgaagaatgcttcaaacg | 780 | |
| 781 | aatcattcggttacactgaagacgaaatcgtttcttctgatgtagttggtatgacttacg | 840 | |
| 841 | gttcattattcgatgcaacacaaacacgtgttatgacagttggcgatcgccaattagtta | 900 | |
| 901 | aagtagcagcttggtatgataacgaaatgtc | | |

(25) Staphylococcus caseolyticus

| | | | |
|---|---|---|---|
| 1 | atggttttggtagaattggtcgtttattagaacagcatgcacgggtaaaagcattggatg | 60 | (SEQ ID NO:25) |
| 61 | cacaatatccagaaattgatatttattcaggtgttgagatggatatattagcagatggcg | 120 | |
| 121 | aaatggattatagcaatgacgtgctagcacagcttgactattgtattggtgctattcatc | 180 | |
| 181 | agtcgttgaatcaaagtgaagatgagattatgaaacgcctgatcaatgcctgtaataatc | 240 | |
| 241 | catacattagacatattgctcatccaacgggaaggttgattggtcgccgtaatggttatc | 300 | |
| 301 | atgtaaacatgccgaaactcatcgaaactgcacagaagacaaataccatccttgaaatca | 360 | |
| 361 | atgcacatccgatgcgccttgatttatcgagcgacgtattaaagcaatatccagatatta | 420 | |
| 421 | aacttgtgatcaacacagacgcgcgtgcaatcgatcagcttgatttaatgaaatatggtg | 480 | |
| 481 | tgggtacagcaataaaaggccatgtgaaaaaagaacaggtaataaacactttaccgcgta | 540 | |
| 541 | aggattttaaatcttggatacagaatgggaagtaattatatgaataaaaaagcattaaat | 600 | |
| 601 | atattagaatataacaaaattattgagcgtgttgacgcatttactcaaaatgaactttca | 660 | |
| 661 | agcaaaaagtgaggatgacacancctatcagcgacaaagctgaaatagatagcatgcttg | 720 | |
| 721 | cacagcttggtatgataacgaaatgtc | | |

(26) Staphylococcus aureus

| | | | |
|---|---|---|---|
| 1 | atggttttggtagaattggtcgtttagcattcagaagaattcaagaagtagaaggtcttg | 60 | (SEQ ID NO:26) |
| 61 | aagttgtagcagtaaacgacttaacagatgacgacatgttagcgcatttattaaaatatg | 120 | |
| 121 | acactatgcaaggtcgtttcacaggtgaagtagaggtagttgatggtggtttccgcgtaa | 180 | |
| 181 | atggtaaagaagttaaatcattcagtgaaccagatgcaagcaaattaccttggaaagact | 240 | |
| 241 | taaatatcgatgtagtgttagaatgtactggtttctacactgataaagataaagcacaag | 300 | |
| 301 | ctcatattgaagcaggcgctaaaaaagtattaatctcagcaccagctactggtgacttaa | 360 | |
| 361 | aaacaatcgtattcaacactaaccaccaagagttagacggttctgaaacagttgtttcag | 420 | |
| 421 | gtgcttcatgtactacaaactcattagcaccagttgctaaagttttaaacgatgactttg | 480 | |
| 481 | gtttagttgaaggtttaatgactacaattcacgcttatacaggtgatcaaaatacacaag | 540 | |
| 541 | acgcacctcacagaaaaggtgacaaacgtcgtgctcgtgcagcagcagaaaacatcatcc | 600 | |
| 601 | ctaactcaacaggtgctgctaaagctatcggtaaagttattcctgaaatcgatggtaaat | 660 | |
| 661 | tagatggtggtgcacaacgtgttcctgtagctacaggttcattaactgaattaacagtag | 720 | |
| 721 | tattagaaaaacaagacgtaacagttgaacaagttaacgaagctatgaaaaatgcttcaa | 780 | |
| 781 | acgaatcattcggttacactgaagacgaaatcgtttcttcagacgttgtaggtatgactt | 840 | |
| 841 | acggttcattatttgacgctacacaaactcgtgtaatgtcagttggcgaccgtcaattag | 900 | |
| 901 | ttaaagttgcagcttggtatgataacgaaatgtc | | |

(27) Staphylococcus warneri

| | | | |
|---|---|---|---|
| 1 | atggttttggtagaattggtcgtttagcatttagaagaattcaagacgtagaaggtttag | 60 | (SEQ ID NO:27) |
| 61 | aagtagttgcagtaaacgacttaactgatgacgatatgttagcacacttattaaaatatg | 120 | |
| 121 | acactatgcaaggtcgcttcactggtgaagtagaagttatcgacggtggattccgtgtta | 180 | |
| 181 | acggtaaggaagttaaatcattcgatgaaccagatgcaagcaaattaccttggaaagact | 240 | |
| 241 | tagatatcgacgtagtattagaatgtactggtttctatactgacaaagataaagcacaag | 300 | |
| 301 | ctcatgatgacgcaggtgctaaaaaagtattaatctcagctccagcaactggtgacttaa | 360 | |
| 361 | aaacaatcgtttacaacactaaccacgatgaattagatggttctgaaacagtagtatcag | 420 | |
| 421 | gtgcatcatgtactactaactcattagctcctgtagctaaagtgttaagtgatgagttcg | 480 | |
| 481 | gtttagttgaaggtttaatgacaactatccacgcttacactggtgaccaaaatactcaag | 540 | |
| 541 | atgctccacacagaaaaggcgacaaacgtcgtgctcgtgctgcagcagaaaacatcatcc | 600 | |
| 601 | ctaactcaactggtgctgctaaagcaatcggtaaagtaattcctgaaatcgacggaaaat | 660 | |
| 661 | tagacggtggtgcacaacgtgttccagttgcaactggttcattaactgaattaacagtag | 720 | |
| 721 | tattagaaaaagacgtaacagttgaacaagttaacgaagcaatgaaacaagcttctgacg | 780 | |
| 781 | aatcattcggttacactgaagacgaaatcgtatcttctgatgtagttggtatgacttacg | 840 | |
| 841 | gttcattattcgatgcaactcaaactcgtgtaatggctgttggtggtcgtcaattagtta | 900 | |
| 901 | aagtagcagcttggtatgataacgaaatgtc | | |

SEQ ID Nos: 1-25 correspond to NCBI GenBank Accession Nos. AF495474-495498, respectively; SEQ ID NO:26 corresponds to nucleotides 1867-2800 of NCBI GenBank Accession No. AJ133520; and SEQ ID NO:27 corresponds to nucleotides 32-962 of NCBI GenBank Accession No. AY024363.

The present invention provides a method for detecting Staphylococcus spp. Specifically, a nucleic acid template from a sample suspected of containing Staphylococcus spp. is amplified with a pair of Staphylococcus spp.-specific primers. The amplification product, if any, is detected by either gel electrophoresis and staining, or by probe hybridization. Detection of an expected amplification product indicates the presence of Staphylococcus spp. in the sample.

The nucleic acid template can be DNA (e.g., a genomic fragment or a restriction fragment) or RNA, in a purified or unpurified form. A nucleic acid template can be obtained from a human or an animal (e.g., a specimen).

The present invention features Staphylococcus spp.-specific primers containing oligo-nucleotides selected from the gap gene region described above. One primer contains an oligo-nucleotide selected from a member of the group consisting of SEQ ID NOs:28-52 (corresponding to SEQ ID NOs: 1-25 but excluding the first 26 and the last 25 nucleotides); the other primer contains an oligo-nucleotide selected from a sequence complementary to the member. Typically, a primer is 14-40 nucleotides in length (PCR Application Manual, Boehringer Mannheim, 1995, page 37). Non-Staphylococcus sequences can be added to the 5′-end of a primer. An example of a non-Staphylococcus sequence is a sequence containing a restriction site, which can be used to facilitate cloning of the amplification product.

The present invention also features Staphylococcus-specific probes chosen from the gap gene region described above, i.e., SEQ ID NOs: 1-27 and their complimentary sequences. These probes can be used for detecting Staphylococcus spp. by hybridizing to an unamplified Staphylococcus nucleic acid or an Staphylococcus nucleic acid amplified with the above-described primer pairs. SEQ ID NOs: 1-27 and their complimentary sequences are examples of such probes.

The probes can be immobilized on the surface of a solid support, such as a membrane (a nylon membrane or a nitrocellulose membrane), a glass, or a plastic polymer. Immobilization of probes to a membrane can be achieved by baking at 80° C. or UV cross-linking. The probes can also be covalently linked to a material (e.g., poly-lysine) coated on the surface of a glass. In addition, a novel method of immobilizing probes on a plastic polymer has recently been developed. See U.S. application Ser. No. 09/906,207. Alternatively, the probes can be synthesized de novo at precise positions on a solid substrate. See Schena et al., 1995, Science 270: 467; Kozal et al., 1996, Nature Medicine 2(7): 753; Cheng et al., 1996, Nucleic Acids Res. 24(2): 380; Lipshutz et al., 1995, BioTechniques 19(3): 442; Pease et al., 1994, Proc. Natl. Acad. Sci. USA 91: 5022; Fodor et al., 1993, Nature 364: 555; and Fodor et al., WO 92/10092.

A target Staphylococcus nucleic acid (e.g., an amplification product described above) can be detected by binding it to an immobilized probe. To facilitate the detection, a labeled amplification product can be generated with a labeled amplification primer. Alternatively, the labeling can be done, chemically or enzymatically, after amplification. Examples of labeling reagents include, but are not limited to, a fluorescent molecule (e.g., fluorescein and rhodamine), a radioactive isotope (e.g., ³²P and 125I), a colorimetric reagent, and a chemiluminescent reagent. Biotin and digoxgenin are frequently used for calorimetric detection on a membrane or a plastic polymer. Fluorescent labels, such as Cy3 and Cy5, are widely used for detection on a glass. In addition, artificial tagging tails (e.g., a protein or its antibody) can be conjugated to the 5′-end of the primers or either end of the probes. See Stetsenko and Gait, 2000, J. Org. Chem. 65(16): 4900.

The specificity of the Staphylococcus spp. detection method of this invention is unexpectedly high. A probe derived from the gap gene region of Staphylococcus intermedius (67-71% identical to 25 other Staphylococcus spp.) detected only genomic DNA from Staphylococcus spp., but not that from other bacteria such as Salmonella spp., E. coli, Shigella spp., Enterobacter aerogenes, Citrobacterfreundii, Klebsiella pneumoniae, Listeria monocytogenes, Vibrio parahaemolyticus, Bacillus cereus, and Streptococcus aglactiae (see Example 2 below). Most unexpected is the ability of a probe derived from a Staphylococcus species to discriminate this Staphylococcus species from other Staphylococcus species having as high as 94% homology in the gap gene region (see Example 1 below).

Also within the scope of this invention is use of Staphylococcus spp.-specific sequences described above in combination with other species-specific nucleic acid sequences for simultaneously identification of multiple microorganisms.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications recited herein are hereby incorporated by reference in their entirety.

### EXAMPLE 1

### Typing Staphylococcus spp. by Probe Hybridization

1. Bacterial Strains

Twenty-seven Staphlylococci strains were obtained from Culture Collection and Research Center (CCRC), Hsin-Chu, Taiwan. The bacterial strains and their registration numbers are listed in TABLE 1.

TABLE 1
LIST OF STAPHYLOCOCCUS STRAINS

| No. | Bacterial Strain | CCRC No. | ATCC No. |
|---|---|---|---|
| 1 | Staphylococcus arlettae | 13975 | 43957 |
| 2 | Staphylococcus auricularis | 13912 | 33753 |
| 3 | Staphylococcus caprae | 13911 | 35538 |
| 4 | Staphylococcus capitis | 12161 | 27840 |
| 5 | Staphylococcus carnosus | 12922 | — |
| 6 | Staphylococcus chromogenes | 12924 | 43764 |
| 7 | Staphylococcus cohni | 12155 | 29974 |
| 8 | Staphylococcus delphini | 15270 | 49171 |
| 9 | Staphylococcus epidermidis | 10783 | 155 |
| 10 | Staphylococcus equorum | 13974 | 43958 |
| 11 | Staphylococcus gallinarum | 13913 | 35539 |
| 12 | Staphylococcus haemolyticus | 12923 | 29970 |
| 13 | Staphylococcus hominis | 12156 | 27844 |
| 14 | Staphylococcus hyricus | 12925 | 11249 |
| 15 | Staphylococcus intermedius | 15235 | 49051 |
| 16 | Staphylococcus kloosii | 13973 | 43959 |
| 17 | Staphylococcus lentus | 12926 | 29070 |
| 18 | Staphylococcus lugdunesis | 13971 | 43809 |
| 19 | Staphylococcus piscifermentans | 15314 | 51337 |
| 20 | Staphylococcus saprophyticus | 10786 | 15305 |
| 21 | Staphylococcus schleiferi | 13972 | 43808 |
| 22 | Staphylococcus sciuri | 12972 | 29062 |
| 23 | Staphylococcus simulans | 10778 | 11631 |
| 24 | Staphylococcus xylosus | 12930 | 29971 |
| 25 | Staphylococcus caseolyticus | | 13548 |
| 26 | Staphylococcus aureus | 10780 | 12600 |
| 27 | Staphylococcus warneri | 12929 | 27836 |

2. Cultivation of Bacterial Strains

One loopful of each test strain was plated on Luria-Bertani agar (LB; 0.5% yeast extract, 1% trypton, 0.5% NaCl, 1.5-2% agar) and incubated for overnight (14 hr) at 37° C. to recover the bacterial vitality. A single colony was picked for each strain, inoculated into 3 ml sterilized LB broth, and incubated for overnight at 37° C. with shaking at 150-180 rpm. The cell density of each bacterial strain was up to ˜1×10¹⁰ cells/ml.

3. Preparation of Bacterial Total Genomic DNA

Total genomic DNA was prepared from 1 ml of bacterial overnight culture. Cells were harvested by centrifugation at 6,000×g for 5 min and discarding the culture supernatant. Cell pellets were resuspended in 50 μl STET buffer (0.1M NaCl, 10 mM Tris-HCl, pH 8.0, 1 mM EDTA, and 5% Triton X-100) containing 5 μl lysozyme (10 mg/ml). The cells were lysed after incubation for 15 min at 37° C. followed by 10 min-boiling. The DNA-containing supernatant were roughly separated from cell debris after 5-min centrifugation, extrated with phenol/chloroform (1:1), and centrifuged at 10,000×g for 10 min. The upper layer of the extraction mixture, ca. 40 μl, was transferred to a new eppendorf tube and used as a DNA template for amplification.

4. Amplification of Bacterial Genomic DNA with Genus-Specific Oligo-Nucleotide Primers

Partial gap gene sequences of 27 different Staphylococcus species (TABLE 1) were analyzed by amplification using a primer pair (Yugueros et al., 2000, J. Clinical Microbiology 38: 4351-4355) located in the conserved region of the gap gene and subsequent sequence analysis. The primers used were GF-1 (5′-atggttttggtagaattggtcgttta-3′) and GR-2 (5′-gacatttcgttatcataccaagctg-3′), both of which were synthesized by GENASIA SCIENTIFICS INC. This pair of primers has been shown to be specific for Staphylococcus (Yugueros et al., 2000, J. Clinical Microbiology 38: 4351-4355).

The amplification reaction mixture (50 μl) contained 5 μl 10× Taq DNA polymerase buffer (Promega, Madison, Wis., USA), 5 μl 25 mM MgCl₂ (Promega), 5 μl 2.5 mM dNTPs (Promega), 1 μl 20 μM of each oligo-nucleotide primer, 1 μl of extracted total genomic DNA, 1 U of Taq DNA polymerase (Promega), and sterilized dH₂O. Amplification was carried out using GeneAmpg® PCR System 2400 (Perkin-Elmer) as followed: 95° C. for 2 min; 30 cycles of 95° C. for 30 sec, 55° C. for 30 sec, 72° C. for 30 sec; and a final extension of 72° C. for 6 min.

5. Cloning and Sequencing of Staphylococcus spp. Partial Gap Gene Fragments

Each of the amplified partial gap gene fragments (ca. 933 bp) was cloned into a pGEM-T Easy vector system (Promega, Madison, Wis., USA), which was transformed into an E. coli bacterial host.

Three E. coli transformants were selected from each transformation, and the plasmid DNA containing the amplified Staphylococcus spp. gap gene fragment was isolated using QIAamp DNA Mini Kit (Qiagen, Hilden, Germany). The presence of the amplified partial gap gene fragment was confirmed first by restriction enzyme digestion and then by sequencing using two primers, T7 promoter primer and SP6 promoter primer (Promega, Madison, Wis., USA). Twenty-five of these gene fragments are newly identified sequences; 2 of them have been previously published (i.e., Staphylococcus aureus gap gene fragment, NCBI GenBank Accession No. AJ133520, nucleotides 1867-2800; and Staphylococcus warneri gap gene fragment, NCBI GenBank Accession No. AY024363, nucleotides 32-962). The plasmids containing the 27 Staphylococcus spp. partial gap gene fragments are listed in TABLE 2 below.

TABLE 2
PLASMIDS CARRYING PARTIAL STAPHYLOCOCCUS
SPP. GAP GENE FRAGMENTS

| No. | Strains | Plasmid Name |
|---|---|---|
| 1 | Staphylococcus aureus | pGAP-A1-1 |
| 2 | Staphylococcus arlettae | pGAP-A1-34 |
| 3 | Staphylococcus auricularia | pGAP-A2-2 |
| 4 | Staphylococcus carnusus | pGAP-A2-5 |
| 5 | Staphylococcus caprae | pGAP-A1-29 |
| 6 | Staphylococcus capitis | pGAP-A1-46 |
| 7 | Staphylococcus caseolyticus | pGAP-A2-37 |
| 8 | Staphylococcus hominis | pGAP-A1-4 |
| 9 | Staphylococcus cohni | pGAP-A1-7 |
| 10 | Staphylococcus chromogenes | pGAP-A2-31 |
| 11 | Staphylococcus delphini | pGAP-A2-19 |
| 12 | Staphylococcus epidermidis | pGAP-A1-32 |
| 13 | Staphylococcus equorum | pGAP-A2-16 |
| 14 | Staphylococcus gallinarum | pGAP-A2-5 |
| 15 | Staphylococcus haemolyticus | pGAP-A1-41 |
| 16 | Staphylococcus hyricus | pGAP-A2-27 |
| 17 | Staphylococcus intermedius | pGAP-A2-38 |
| 18 | Staphylococcus kloosii | pGAP-A2-14 |
| 19 | Staphylococcus lentus | pGAP-A1-17 |
| 20 | Staphylococcus lugdunesis | pGAP-A2-9 |
| 21 | Staphylococcus piscifermentans | pGAP-A2-20 |
| 22 | Staphylococcus saprophyticus | pGAP-A1-38 |
| 23 | Staphylococcus sciuri | pGAP-A1-50 |
| 24 | Staphylococcus schleifer | pGAP-A2-11 |
| 25 | Staphylococcus simulans | pGAP-A1-22 |
| 26 | Staphylococcus warneri | pGAP-A1-19 |
| 27 | Staphylococcus xylosus | pGAP-A1-24 |

6. Sequence Alignment of Amplified Partial Gap Gene Fragments

Classification of homology degree of these bacteria is based on sequence analysis results obtained by using the phylogenetic tree and sequence pair distance program (Lesegene, DNAstar Inc., Wis., USA) and the BLAST sequence alignment program (NCBI GenBank).

The homology degree among 26 of the Staphylococcus spp. is in the range of 68-99%. One exception is the strain Staphylococcus caseolyticus, which shows only 22-27% homology to the other 26 Staphylococcus spp. This strain has been previously misidentified as Micrococcus caseolyticus (Schleifer et al., 1982, J. Systematic Bacteriology 32: 15-20). DNA-DNA hybridization data and other physiological and biochemical data have been used to reclassify this bacterium. Thirteen pairs of Staphylococcus spp. (TABLE 3) have a homology degree over 90%. In particular, pair no. 1 (i.e., Staphylococcus epidermidis-Staphylococcus warneri) shows 99% homology, and the other 12 pairs have a homology degree up to 94%.

TABLE 3
HOMOLOGY DEGREES FOR THIRTEEN
PAIRS OF STAPHYLOCOCCUS SPP.

| Pair | | | Homology |
|---|---|---|---|
| No. | Bacterial Strain-1 | Bacterial Strain-2 | Degree* |
| 1 | Staphylococcus | Staphylococcus | 99.5% |
| | epidermidis | warneri | |
| 2 | Staphylococcus | Staphylococcus | 94% |
| | capitis | caprae | |
| 3 | Staphylococcus | Staphylococcus | 94.3% |
| | camusus | hyricus | |
| 4 | Staphylococcus | Staphylococcus | 93.8% |
| | camusus | simulans | |
| 5 | Staphylococcus | Staphylococcus | 92.3% |
| | saprophyticus | xylosus | |
| 6 | Staphylococcus | Staphylococcus | 92.3% |
| | lentus | sciuri | |
| 7 | Staphylococcus | Staphylococcus | 91.3% |
| | wareri | capitis | |
| 8 | Staphylococcus | Staphylococcus | 91.2% |
| | hyricus | chromogenes | |
| 9 | Staphylococcus | Staphylococcus | 91.1% |
| | epidermidis | capitis | |
| 10 | Staphylococcus | Staphylococcus | 90.5% |
| | saprophyticus | equorum | |
| 11 | Staphylococcus | Staphylococcus | 90.5% |
| | arlettae | kloosii | |
| 12 | Staphylococcus | Staphylococcus | 90.3% |
| | caprae | wareri | |
| 13 | Staphylococcus | Staphylococcus | 90.1% |
| | caprae | epidermis | |

*Degree of homology between two species is determined by using either the DNA sequence analyzing software Lesegene (i.e., pair nos 3-13) or GenBank BLAST (i.e., pair nos 1 and 2).

7. Preparation of Hybridization Probes and Simulative Target DNA

Both hybridization probes and simulative target DNA were generated by the amplifying the gap gene of three bacteria, Staphylococcus aureus, Staphylococcus caprae, and Staphylococcus capitis, using the primer pair GF-1 and GR-2. The amplified partial gap gene fragments from Staphylococcus caprae and from Staphylococcus capitis are 94% identical, while each of them shows 89.2% homology to the Staphylococcus aureus gap gene fragment. The amplification products (around 930 bp) obtained by using biotin-labeled primers were used as simulative target DNA, while the amplification products obtained by using unlabeled primers were used as hybridization probes. The amplification was carried out as described above, except that around 100 ng purified plasmid DNA was used as DNA template instead of bacterial genomic DNA. The amplified products were extracted with phenol/chloroform (v/v=1/1) and precipitated with ethanol to remove excess primers, dNTPs and the enzyme.

8. Hybridization

The three hybridization probes were dissolved in a probe solution (DR. Probsol, DR. Chip Biotechnology Inc.) to a final concentration of 20 ng/μl, spotted and immobilized on a solid support (DR. Chip Biotechnology Inc.).

Four microliters of each biotin-labeled simulative target DNA (stock concentration: 40 ng/μl) were mixed with 500 μl hybridization buffer (Dr. Hyb™ buffer, DR. Chip Biotechnology Inc.). The DNA mixture was boiled for 5 min, chilled, applied to the solid support, and incubated at 80° C. for 1 hr with shaking.

Three wash steps were carried out to eliminate unspecific binding. The solid support was first washed with a wash buffer (DR. Wash from DR. Chip Biotechnology Inc., Taiwan) for at least three times at room temperature. A stringent wash was then performed with the same wash buffer at 80° C. for 30 min with shaking. The solid support was finally washed with the same wash buffer for at least three times at room temperature again.

Biotin-specific calorimetric detection was performed by incubating the solid support in a Blocking Reagent (Roche) containing alkaline phosphatase-conjugated streptavidin (Promega). The solid support was subsequently washed three times with the wash buffer, and incubated with NBT/BCIP solution (Roche) diluted with a detection buffer in a ratio recommended by the supplier for about 10 minutes in dark.

Unexpectedly, the Staphylococcus aureus target DNA only hybridized to the probe originated from the same bacterial species, but not to the probes originated from the other two Staphylococcus species. Similarly, the Staphylococcus caprae and Staphylococcus capitis target DNAs only hybridized to the corresponding probe. This result indicates that this method can be used to differentiate Staphylococcus spp. strains with very high homology degree (e.g., 94%).

### EXAMPLE 2

### Detecting Staphylococcus Genus by Probe Hybridization

1. Bacterial Strains

Eighty food-borne bacterial strains listed in TABLE 4 were used in this study. Group I contains 10 Staphylococcus spp.; Group II contains another 10 Staphylococcus spp.; Group III contains 10 Salmonella spp.; Group IV contains 30 E. coli strains, including non-pathogenic strains and pathogenic strains such as ETEC, EIEC, EPEC, EAggEC and EHEC; and Group V contains other bacterial strains, including 11 pathogenic strains (i.e., 4 Shigella spp.; 3 coliform bacterial strains Enterobacter aerogenes, Citrobacterfreundii, and Klebsiella pneumoniae; 3 other food-borne pathogenic bacterial strains Listeria monocytogenes, Vibrio parahaemolyticus, and Bacillus cereus; and 1 infectious bacterial strain causing cattle mastitis, Streptococcus agalactiae). These bacterial strains were obtained from different sources, i.e., Culture Collection and Research Center (CCRC), Hsin-Chu, Taiwan; American Type Culture Collection (ATCC), Rockville, Md., USA; United States Department of Agriculture (USDA), Washington, D.C., USA; Department of Food Science, National Chung-Hsing University (NCHU; Taichung, Taiwan, R.O.C.); Pingtung University of Technology (PT), Pingtung, Taiwan; and Deutsche Sammlung von Mikroorganismen und Zellkulturen GmbH (DSMZ; Braunschweig, Germany).

TABLE 4
BACTERIAL STRAINS

| Group | Strains | No. | Source & Number | |
|---|---|---|---|---|
| Group I | Staphylococcus | 1 | CCRC 13975 | ATCC 43957 |
| Staphylococcus | arlettae | | | |
| | Staphylococcus | 1 | CCRC 13912 | ATCC 33753 |
| | auricularia | | | |
| | Staphylococcus | 1 | CCRC 12924 | ATCC 43764 |
| | chromogenes | | | |
| | Staphylococcus | 1 | CCRC 10783 | ATCC 155 |
| | epidermidis | | | |
| | Staphylococcus | 1 | CCRC 13913 | ATCC 35539 |
| | gallinarum | | | |
| | Staphylococcus | 1 | CCRC 12156 | ATCC 27844 |
| | hominis | | | |
| | Staphylococcus | 1 | CCRC 12926 | ATCC 29070 |
| | lentus | | | |
| | Staphylococcus | 1 | CCRC 15314 | ATCC 51337 |
| | piscifermentans | | | |
| | Staphylococcus | 1 | CCRC 10778 | ATCC 11631 |
| | simulans | | | |
| | Staphylococcus | 1 | CCRC 13972 | ATCC 43808 |
| | schleiferi | | | |
| | Staphylococcus | 1 | CCRC 12930 | ATCC 29971 |
| | xylosus | | | |
| | Staphylococcus | 1 | CCRC 12161 | ATCC 49324 |
| | capitis | | | |
| | Staphylococcus | 1 | CCRC 12922 | DSM 20501 |
| | carnusus | | | |
| Group II | Staphylococcus | 1 | CCRC 12155 | ATCC 29974 |
| Staphylococcus | cohni | | | |
| | Staphylococcus | 1 | CCRC 13974 | ATCC 43953 |
| | equorum | | | |
| | Staphylococcus | 1 | CCRC 12923 | ATCC 29970 |
| | haemolyticus | | | |
| | Staphylococcus | 1 | CCRC 12925 | ATCC 11249 |
| | hyricus | | | |
| | Staphylococcus | 1 | CCRC 13973 | ATCC 43959 |
| | kloosii | | | |
| | Staphylococcus | 1 | CCRC 13971 | ATCC 43809 |
| | lugdunesis | | | |
| | Staphylococcus | 1 | CCRC 10786 | ATCC 15305 |
| | saprophyticus | | | |
| | Staphylococcus | 1 | CCRC 12927 | ATCC 29062 |
| | sciuri | | | |
| | Staphylococcus | 1 | CCRC 12929 | ATCG 27336 |
| | warneri | | | |
| | Staphylococcus | 1 | CCRC 15270 | ATCC 49171 |
| | delphini | | | |
| | Staphylococcus | 1 | CCRC 15263 | ATCC 13543 |
| | caseolyticus | | | |
| Group III | Salmonella | 1 | CCRC 14875 | ATCC 9992V |
| Salmonella | typhi | | | |
| spp. | Salmonella | 1 | CCRC 10747 | ATCC 14028 |
| | typhiurium | | | |
| | Salmonella | 1 | CCRC 15450 | ATCC 6956 |
| | salamae | | | |
| | Salmonella | 1 | CCRC 14878 | ATCC 9150 |
| | paratyphi A | | | |
| | Salmonella | 1 | CCRC 15454 | ATCC 23201 |
| | california | | | |
| | Salmonella | 1 | CCRC 10744 | ATCC 13076 |
| | enteritidis | | | |
| | Salmonella | 1 | CCRC 15455 | ATCC 19128 |
| | etterbeele | | | |
| | Salmonella | 1 | CCRC 15453 | ATCC 15782 |
| | postsdam | | | |
| | Salmonella | 1 | NCHU | USDA |
| | aberdeen | | | |
| | Salmonella | 1 | NCHU | USDA |
| | albany | | | |
| | Salmonella | 1 | NCHU | USDA |
| | amger | | | |
| | Salmonella | 1 | NCHU | PT |
| | anatum | | | |
| Group IV | EAggEC | 4 | NCHU | — |
| E. coli | ETEC | 4 | NCHU | — |
| | EHEC | 5 | NCHU | — |
| | EPEC | 5 | NCHU | — |
| | EIEC | 3 | NCHU | — |
| | Non- | 9 | NCHU | — |
| | pathogenic | | | |
| Group V | Streptococcus | 1 | CCRC10787 | ATCC13813 |
| Other strains | agalactea | | | |
| | Bacillus cereus | 1 | CCRC11827 | ATCC25428 |
| | Shigella | 1 | CCRC13983 | ATCC13313 |
| | dysenteria | | | |
| | Shigella boydii | 1 | CCRC15961 | ATCC8704 |
| | Shigella | 1 | CCRC10772 | ATCC12022 |
| | flexneri | | | |
| | Shigella sonnei | 1 | CCRC10773 | ATCC9290 |
| | Vibrio para- | 1 | CCRC10806 | ATCC17802 |
| | haemolyticus | | | |
| | Listeria | 1 | CCRC14930 | — |
| | monocytogenes | | | |
| | Enterobacter | 1 | CCRC10370 | ATCC13048 |
| | aerogenes | | | |
| | Citrobacter | 1 | CCRC12291 | ATCC8090 |
| | freundii | | | |
| | Klebsiella | 1 | CCRC15627 | — |
| | pneumoniae | | | |
| Positive control | Staphylococcus | 1 | CCRC10780 | ATCC12600 |
| | aureus | | | |
| strains | Staphylococcus | 1 | CCRC13911 | ATCC35538 |
| | caprae | | | |
| | Staphylococcus | 1 | CCRC15235 | ATCC49051 |
| | intermedius | | | |

2. Cultivation of Bacterial Strains

One loop of each test strain was plated on Luria-Bertani agar (LB; 0.5% yeast extract, 1% trypton, 0.5% NaCl, 1.5-2% agar) and incubated for overnight (14 hr) at 37° C. A single colony was picked for each strain and inoculated into 10 ml sterilized LB broth. Bacterial cultures were incubated for overnight at 37° C. with shaking at 120-150 rpm.

3. Preparation of Bacterial Mixtures and Extraction of Genomic DNA

One milliliter suspension (10⁸-10¹⁰ cells) was taken from each bacterial culture and mixed in one tube according to the groups listed in TABLE 1. QIAamp DNA Mini Kit (Qiagen, Hilden, Germany) was used for extraction of total genomic DNA from 1 ml mixed culture and for further purification. Quantification of extracted genomic DNA was carried out by spectrophotometric method (V-530, Jasco, Jascon International Co., LTD, Japan). The final concentration of the genomic DNA prepared from each bacterial mixture was adjusted to 5 μg/μl.

4. Immobilization of DNA on a Solid Support

One microliter (5 μg/μl) of prepared DNA (equivalent to a DNA extract from 4.1×10⁵-1.7×10⁸ cells for each bacterial strain), including mixed genomic DNA of each bacterial group, genomic DNA of a certain bacteria species, and control plasmid DNA, was dissolved in a probe solution (DR. Probsol, DR. Chip Biotechnology Inc., Taiwan), denatured at 95° C. for 5 min, and chilled immediately on ice. The pretreated DNA solution was dotted and cross-linked on a nylon membrane (Hybond™-N, Amersham Pharmacia Biotech) of 5 cm×5 cm.

5. Preparation of Biotin-Labeled Detection Probes

Biotin-labeled probes were generated as described above. Plasmid pGAP-A2-38 containing the Staphylococcus intermedius gap gene fragment was used as a DNA template for amplification. Among the 26 Staphylococcus spp. (Staphylococcus caseolyticus excluded), Staphylococcus intermedius shows the lowest homology (67%-71%) to other species.

6. Hybridization

The biotin-labeled detection probe was dissolved in 50 ml hybridization buffer (Dr. Hyb™ buffer, DR. Chip Biotechnology Inc.) to a final concentration of 0.1 nM, denatured at 95° C. for 5 min, and immediately chilled on ice for 5 min. This pretreated detection probe solution was then hybridized to DNA spotted on a nylon membrane for 12 hr at 50° C. The nylon membrane was then washed with 50 ml wash buffer (DR. Wash from DR. Chip Biotechnology Inc., Taiwan) twice at 50° C. for 5 min.

Unexpectedly, positive signals were detected at spots containing the mixed genomic DNA of Group I and II bacteria, the genomic DNA of three Staphylococcus spp. (i.e., Staphylococcus aureus, Staphylococcus caprae, and Staphylococcus intermedius), and positive controls. No signal was detected at spots containing the mixed genomic DNA of Group III, IV, or V bacteria. This result indicates that the Staphylococcus spp. gap gene fragment can be used to differentiate Staphylococcus from other bacterial genera.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A novel nucleic acid comprising an oligo-nucleotide selected from a member of the group consisting of SEQ ID NOs: 1-25 and sequences complementary to SEQ ID NOs:1-25, wherein the nucleic acid is 10-1000 nucleotides in length.

2. The nucleic acid of claim 1, wherein the nucleic acid is 10-500 nucleotides in length.

3. The nucleic acid of claim 2, wherein the nucleic acid is 10-200 nucleotides in length.

4. The nucleic acid of claim 3, wherein the nucleic acid is 10-50 nucleotides in length.

5. The nucleic acid of claim 4, wherein the nucleic acid is 10-20 nucleotides in length.

6. The nucleic acid of claim 1, wherein the oligo-nucleotide is a member of the group consisting of SEQ ID NOs:1-25 and sequences complementary to SEQ ID NOs:1-25.

7. A pair of amplification primers, comprising
a first primer containing a first oligo-nucleotide selected from a member of the group consisting of SEQ ID NOs:28-52, and
a second primer containing a second oligo-nucleotide selected from a sequence complementary to the member,
wherein each primer is 14-40 nucleotides in length.

8. The pair of primers of claim 7, wherein each primer is 14-30 nucleotides in length.

9. The pair of primers of claim 8, wherein each primer is 14-20 nucleotides in length.

10. A method of detecting a target Staphylococcus species, comprising:
providing a sample having a nucleic acid from an unknown microorganism;
amplifying the nucleic acid with a pair of primers containing a first primer including a first oligo-nucleotide selected from a member of the group consisting of SEQ ID NOs:28-52 and a second primer including a second oligo-nucleotide selected from a sequence complementary to the member, each primer being 14-40 nucleotides in length; and
detecting an amplification product;
whereby detection of the amplification product indicates the presence of the target Staphylococcus species.

11. The method of claim 10, wherein each primer is 14-30 nucleotides in length.

12. The method of claim 11, wherein each primer is 14-20 nucleotides in length.

13. The method of claim 10, wherein the detecting step includes hybridizing the amplification product to a nucleic acid probe that is 10-1000 nucleotides in length and contains a sequence selected from a member of the group consisting of SEQ ID NOs: 1-27 and sequences complementary to SEQ ID NOs:1-27.

14. The method of claim 13, wherein the nucleic acid probe is 10-500 nucleotides in length.

15. The method of claim 14, wherein the nucleic acid probe is 10-200 nucleotides in length.

16. The method of claim 15, wherein the nucleic acid probe is 10-50 nucleotides in length.

17. The method of claim 16, wherein the nucleic acid probe is 10-20 nucleotides in length.

18. The method of claim 17, wherein the nucleic acid probe is a member of the group consisting of SEQ ID NOs:1-27 and sequences complementary to SEQ ID NOs:1-27.
